(19) United States
(12) Patent Application Publication
(10) Pub. No.: US 2024/0090848 A1
(43) Pub. Date: Mar. 21, 2024

(54) MACHINE LEARNING SIGNAL PROCESSING TECHNIQUES FOR GENERATING PHYSIOLOGICAL PREDICTS

(71) Applicant: UnitedHealth Group Incorporation, Minnetonka, MN (US)

(72) Inventors: Sankalita SAHA, Saratoga, CA (US); Clark N. CALLAHAN, Minneapolis, MN (US); Nels THOMPSON, Edina, MN (US); Ze Yuan LI, New York, NY (US); Cody J. JAZDZEWSKI, Minneapolis, MN (US); Rony KRELL, Brooklyn, NY (US); Tanzeem Yuan CHOUDHURY, New York, NY (US)

(21) Appl. No.: 18/470,468

(22) Filed: Sep. 20, 2023

Related U.S. Application Data
(60) Provisional application No. 63/376,457, filed on Sep. 21, 2022.
(60) Provisional application No. 63/516,028, filed on Jul. 27, 2023.

(51) Int. Cl. A61B 5/00; A61B 5/145; G16H 20/17; G16H 50/20

(52) U.S. Cl. CPC A61B 5/7275; A61B 5/14532; A61B 5/6802; A61B 5/7267; G16H 20/17; G16H 50/20

(57) ABSTRACT

Various embodiments of the present disclosure provide signal interpretation and data aggregation techniques for generating predictive insights for a user. The techniques may include receiving initial physiological features for a user that are based on recorded sensor values for the user. The techniques include generating activity encodings for the user based on interaction data objects for the user and generating a combined input feature vector by aggregating the initial physiological features and the activity encodings. The techniques include generating, using a machine learning model, a physiological prediction for the user based on the combined input feature vector.

### CROSS-REFERENCE TO RELATED APPLICATIONS

This application claims the benefit of U.S. Provisional Application No. 63/376,457, entitled “CGM Signal Processing for Determination of Treatment Effectiveness,” and filed Sep. 21, 2022 and U.S. Provisional Application No. 63/516,028, entitled “CGM Signal Processing for Determination of Treatment Effectiveness,” and filed Jul. 27, 2023, the entire contents of which are hereby incorporated by reference.

### BACKGROUND

Various embodiments of the present disclosure address technical challenges related to complex signal processing techniques given limitations of existing predictive data analysis processes. Traditionally, signal processing techniques are deployed for processing physiological signals, such as glucose readings, to generate real time insights for a user. These insights may be leveraged to inform diagnoses and potential treatments for the user. However, conventional techniques for interpreting signals lack predictive accuracy for anticipating changes to a user's physiological signals over time. Thus, it is traditionally unknown which of many treatments would be most effective for a particular user. Additionally, there is traditionally a significant delay between the start of a treatment and when it is known whether or not it was effective. The combination of many treatment options, the uncertainty of treatment selection, and delayed knowledge of treatment effectiveness may lead to a long, expensive series of trial-and-error experiments with different treatments before an effective treatment is found.

Various embodiments of the present disclosure make important contributions to various existing signal interpretation techniques by addressing each of these technical challenges.

### BRIEF SUMMARY

Various embodiments of the present disclosure disclose signal interpretation techniques for predicting future physiological signals for a user based on physiological and historical features of a user. To do so, some embodiments of the present disclosure leverage a combined input feature vector that aggregates insights from sensor values recorded by CGM devices as well as insights from interaction data for a user to generate predictions through machine learning model. The combined input feature vector enables accurate future predictions for a user that is based on less initial sensor measurements, thereby enabling the generation of predictive insights with less predictive signals. This, in turn, may be practically applied in a clinical setting to more quickly determine whether a treatment is effective, thereby speeding the evaluation of different treatment alternatives.

In some embodiments, a computer-implemented method includes receiving, by one or more processors, one or more initial physiological features for a user that are based on a plurality of recorded sensor values for the user; generating, by the one or more processors, one or more activity encodings for the user based on a plurality of interaction data objects for the user; generating, by the one or more processors, a combined input feature vector for the user by aggregating the one or more initial physiological features and the one or more activity encodings; generating, by the one or more processors and using a machine learning model, a physiological prediction for the user based on the combined input feature vector; and initiating, by the one or more processors, the performance of a prediction-based action based on the physiological prediction.

In some embodiments, a computing system includes memory and one or more processors communicatively coupled to the memory, the one or more processors are configured to receive one or more initial physiological features for a user that are based on a plurality of recorded sensor values for the user; generate one or more activity encodings for the user based on a plurality of interaction data objects for the user; generate a combined input feature vector for the user by aggregating the one or more initial physiological features and the one or more activity encodings; generate, using a machine learning model, a physiological prediction for the user based on the combined input feature vector; and initiate the performance of a prediction-based action based on the physiological prediction.

In some embodiments, one or more non-transitory computer-readable storage media include instructions that, when executed by one or more processors, cause the one or more processors to receive one or more initial physiological features for a user that are based on a plurality of recorded sensor values for the user; generate one or more activity encodings for the user based on a plurality of interaction data objects for the user; generate a combined input feature vector for the user by aggregating the one or more initial physiological features and the one or more activity encodings; generate, using a machine learning model, a physiological prediction for the user based on the combined input feature vector; and initiate the performance of a prediction-based action based on the physiological prediction.

### BRIEF DESCRIPTION OF DRAWINGS

FIG. 1 illustrates an example computing system in accordance with one or more embodiments of the present disclosure.

FIG. 2 is a schematic diagram showing a system computing architecture in accordance with one or more embodiments of the present disclosure.

FIG. 3 is a dataflow diagram showing example data structures and modules for interpreting short term physiological signals in accordance with some embodiments discussed herein.

FIG. 4 is a dataflow diagram showing example data structures and modules for training a machine learning model to interpret short term physiological signals in accordance with some embodiments discussed herein.

FIG. 5 is a flowchart showing an example of a process for interpreting short term physiological signals in accordance with some embodiments discussed herein.

### DETAILED DESCRIPTION

Various embodiments of the present disclosure are described more fully hereinafter with reference to the accompanying drawings, in which some, but not all embodiments of the present disclosure are shown. Indeed, the present disclosure may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “example” are used to be examples with no indication of quality level. Terms such as “computing,” “determining,” “generating,” and/or similar words are used herein interchangeably to refer to the creation, modification, or identification of data. Further, “based on,” “based at least in part on,” “based at least on,” “based upon,” and/or similar words are used herein interchangeably in an open-ended manner such that they do not indicate being based only on or based solely on the referenced element or elements unless so indicated. Like numbers refer to like elements throughout. Moreover, while certain embodiments of the present disclosure are described with reference to predictive data analysis, one of ordinary skills in the art will recognize that the disclosed concepts may be used to perform other types of data analysis.

### I. Computer Program Products, Methods, and Computing Entities

Embodiments of the present disclosure may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query, or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together, such as in a particular directory, folder, or library. Software components may be static (e.g., pre-established, or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In some embodiments, a non-volatile computer-readable storage medium may include a floppy disk, flexible disk, hard disk, solid-state storage (SSS) (e.g., a solid-state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In some embodiments, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for, or used in addition to, the computer-readable storage media described above.

As should be appreciated, various embodiments of the present disclosure may also be implemented as methods, apparatuses, systems, computing devices, computing entities, and/or the like. As such, embodiments of the present disclosure may take the form of an apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises combination of computer program products and hardware performing certain steps or operations.

Embodiments of the present disclosure are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatuses, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some example embodiments, retrieval, loading, and/or execution may be performed in parallel such that multiple instructions are retrieved, loaded, and/or executed together. Thus, such embodiments may produce specifically configured machines performing the steps or operations specified in the block diagrams and flowchart illustrations. Accordingly, the block diagrams and flowchart illustrations support various combinations of embodiments for performing the specified instructions, operations, or steps.

### II. Example Framework

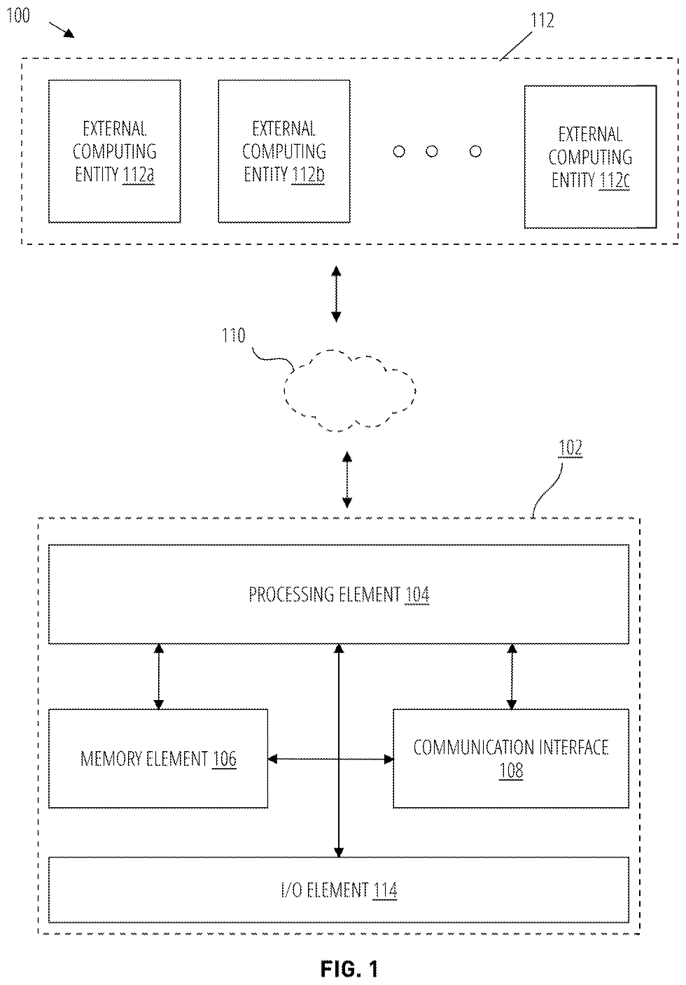

FIG. 1 illustrates an example computing system 100 in accordance with one or more embodiments of the present disclosure. The computing system 100 may include a predictive computing entity 102 and/or one or more external computing entities 112a-c communicatively coupled to the predictive computing entity 102 using one or more wired and/or wireless communication techniques. The predictive computing entity 102 may be specially configured to perform one or more steps/operations of one or more techniques described herein. In some embodiments, the predictive computing entity 102 may include and/or be in association with one or more mobile device(s), desktop computer(s), laptop(s), server(s), cloud computing platform(s), and/or the like. In some example embodiments, the predictive computing entity 102 may be configured to receive and/or transmit one or more datasets, objects, and/or the like from and/or to the external computing entities 112a-c to perform one or more steps/operations of one or more techniques (e.g., signal interpretation techniques, prediction techniques, data interpretation techniques, training techniques, and/or the like) described herein.

The external computing entities 112a-c, for example, may include and/or be associated with one or more entities that may be configured to receive, store, manage, and/or facilitate datasets that include labeled training data, interaction data objects, recorded sensor values, physiological features, activity encodings, combined input feature vectors, and/or the like. The external computing entities 112a-c may provide the input data, such as physiological features, activity encodings, interaction data objects, and/or the like to the predictive computing entity 102 which may leverage the input data to generate combined input feature vectors, physiological prediction, and/or the like. By way of example, the predictive computing entity 102 may include a predictive machine learning model that is configured to leverage physiological and historical data to generate predictive insights for a user. In some examples, the input data may include an aggregation of data from across the external computing entities 112a-c into one or more combined input feature vectors. The external computing entities 112a-c, for example, may be associated with one or more data repositories, cloud platforms, compute nodes, organizations, and/or the like, that may be individually and/or collectively leveraged by the predictive computing entity 102 to obtain and aggregate data for a prediction domain.

The predictive computing entity 102 may include, or be in communication with, one or more processing elements 104 (also referred to as processors, processing circuitry, digital circuitry, and/or similar terms used herein interchangeably) that communicate with other elements within the predictive computing entity 102 via a bus, for example. As will be understood, the predictive computing entity 102 may be embodied in a number of different ways. The predictive computing entity 102 may be configured for a particular use or configured to execute instructions stored in volatile or non-volatile media or otherwise accessible to the processing element 104. As such, whether configured by hardware or computer program products, or by a combination thereof, the processing element 104 may be capable of performing steps or operations according to embodiments of the present disclosure when configured accordingly.

In one embodiment, the predictive computing entity 102 may further include, or be in communication with, one or more memory elements 106. The memory element 106 may be used to store at least portions of the databases, database instances, database management systems, data, applications, programs, program modules, scripts, source code, object code, byte code, compiled code, interpreted code, machine code, executable instructions, and/or the like being executed by, for example, the processing element 104. Thus, the databases, database instances, database management systems, data, applications, programs, program modules, scripts, source code, object code, byte code, compiled code, interpreted code, machine code, executable instructions, and/or the like, may be used to control certain aspects of the operation of the predictive computing entity 102 with the assistance of the processing element 104.

As indicated, in one embodiment, the predictive computing entity 102 may also include one or more communication interfaces 108 for communicating with various computing entities, e.g., external computing entities 112a-c, such as by communicating data, content, information, and/or similar terms used herein interchangeably that may be transmitted, received, operated on, processed, displayed, stored, and/or the like.

The computing system 100 may include one or more input/output (I/O) element(s) 114 for communicating with one or more users. An I/O element 114, for example, may include one or more user interfaces for providing and/or receiving information from one or more users of the computing system 100. The I/O element 114 may include one or more tactile interfaces (e.g., keypads, touch screens, etc.), one or more audio interfaces (e.g., microphones, speakers, etc.), visual interfaces (e.g., display devices, etc.), and/or the like. The I/O element 114 may be configured to receive user input through one or more of the user interfaces from a user of the computing system 100 and provide data to a user through the user interfaces.

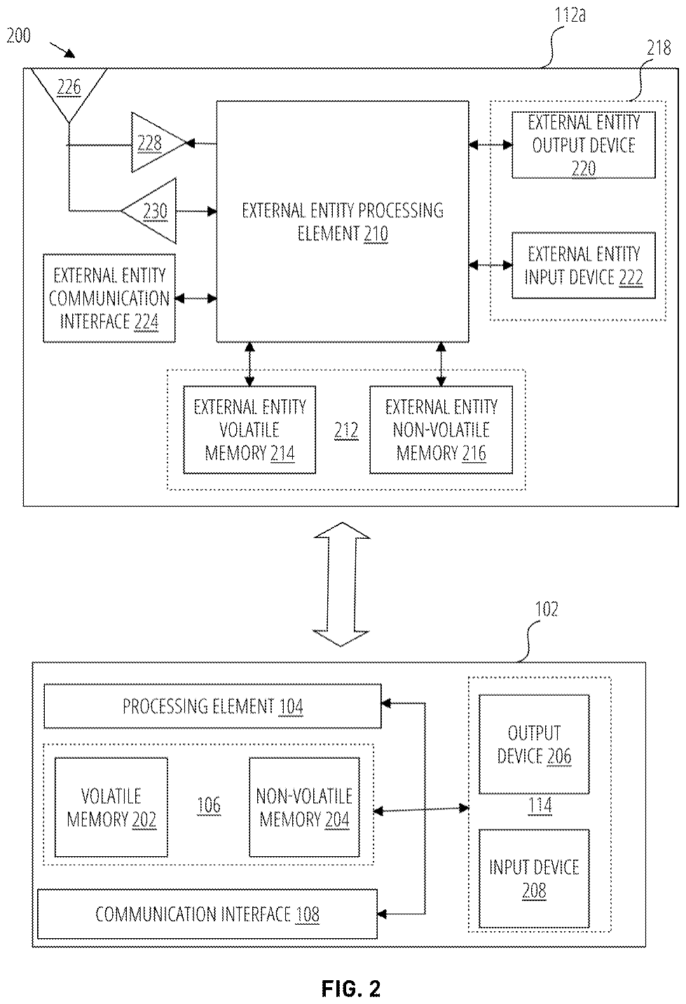

FIG. 2 is a schematic diagram showing a system computing architecture 200 in accordance with some embodiments discussed herein. In some embodiments, the system computing architecture 200 may include the predictive computing entity 102 and/or the external computing entity 112a of the computing system 100. The predictive computing entity 102 and/or the external computing entity 112a may include a computing apparatus, a computing device, and/or any form of computing entity configured to execute instructions stored on a computer-readable storage medium to perform certain steps or operations.

The predictive computing entity 102 may include a processing element 104, a memory element 106, a communication interface 108, and/or one or more I/O elements 114 that communicate within the predictive computing entity 102 via internal communication circuitry, such as a communication bus and/or the like.

The processing element 104 may be embodied as one or more complex programmable logic devices (CPLDs), microprocessors, multi-core processors, coprocessing entities, application-specific instruction-set processors (ASIPs), microcontrollers, and/or controllers. Further, the processing element 104 may be embodied as one or more other processing devices or circuitry including, for example, a processor, one or more processors, various processing devices, and/or the like. The term circuitry may refer to an entirely hardware embodiment or a combination of hardware and computer program products. Thus, the processing element 104 may be embodied as integrated circuits, application specific integrated circuits (ASICs), field programmable gate arrays (FPGAs), programmable logic arrays (PLAs), hardware accelerators, digital circuitry, and/or the like.

The memory element 106 may include volatile memory 202 and/or non-volatile memory 204. The memory element 106, for example, may include volatile memory 202 (also referred to as volatile storage media, memory storage, memory circuitry, and/or similar terms used herein interchangeably). In one embodiment, a volatile memory 202 may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for, or used in addition to, the computer-readable storage media described above.

The memory element 106 may include non-volatile memory 204 (also referred to as non-volatile storage, memory, memory storage, memory circuitry, and/or similar terms used herein interchangeably). In one embodiment, the non-volatile memory 204 may include one or more non-volatile storage or memory media, including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

In one embodiment, a non-volatile memory 204 may include a floppy disk, flexible disk, hard disk, solid-state storage (SSS) (e.g., a solid-state drive (SSD)), solid state card (SSC), solid state module (SSM), enterprise flash drive, magnetic tape, or any other non-transitory magnetic medium, and/or the like. A non-volatile memory 204 may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile memory 204 may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

As will be recognized, the non-volatile memory 204 may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity-relationship model, object model, document model, semantic model, graph model, and/or the like.

The memory element 106 may include a non-transitory computer-readable storage medium for implementing one or more aspects of the present disclosure including as a computer-implemented method configured to perform one or more steps/operations described herein. For example, the non-transitory computer-readable storage medium may include instructions that when executed by a computer (e.g., processing element 104), cause the computer to perform one or more steps/operations of the present disclosure. For instance, the memory element 106 may store instructions that, when executed by the processing element 104, configure the predictive computing entity 102 to perform one or more steps/operations described herein.

Embodiments of the present disclosure may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language, such as an assembly language associated with a particular hardware framework and/or operating system platform. A software component comprising assembly language instructions may require conversion into executable machine code by an assembler prior to execution by the hardware framework and/or platform. Another example programming language may be a higher-level programming language that may be portable across multiple frameworks. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query, or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together, such as in a particular directory, folder, or library. Software components may be static (e.g., pre-established, or fixed) or dynamic (e.g., created or modified at the time of execution).

The predictive computing entity 102 may be embodied by a computer program product includes non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media such as the volatile memory 202 and/or the non-volatile memory 204.

The predictive computing entity 102 may include one or more I/O elements 114. The I/O elements 114 may include one or more output devices 206 and/or one or more input devices 208 for providing and/or receiving information with a user, respectively. The output devices 206 may include one or more sensory output devices, such as one or more tactile output devices (e.g., vibration devices such as direct current motors, and/or the like), one or more visual output devices (e.g., liquid crystal displays, and/or the like), one or more audio output devices (e.g., speakers, and/or the like), and/or the like. The input devices 208 may include one or more sensory input devices, such as one or more tactile input devices (e.g., touch sensitive displays, push buttons, and/or the like), one or more audio input devices (e.g., microphones, and/or the like), and/or the like.

In addition, or alternatively, the predictive computing entity 102 may communicate, via a communication interface 108, with one or more external computing entities such as the external computing entity 112a. The communication interface 108 may be compatible with one or more wired and/or wireless communication protocols.

For example, such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. In addition, or alternatively, the predictive computing entity 102 may be configured to communicate via wireless external communication using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.9 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

The external computing entity 112a may include an external entity processing element 210, an external entity memory element 212, an external entity communication interface 224, and/or one or more external entity I/O elements 218 that communicate within the external computing entity 112a via internal communication circuitry, such as a communication bus and/or the like.

The external entity processing element 210 may include one or more processing devices, processors, and/or any other device, circuitry, and/or the like described with reference to the processing element 104. The external entity memory element 212 may include one or more memory devices, media, and/or the like described with reference to the memory element 106. The external entity memory element 212, for example, may include at least one external entity volatile memory 214 and/or external entity non-volatile memory 216. The external entity communication interface 224 may include one or more wired and/or wireless communication interfaces as described with reference to communication interface 108.

In some embodiments, the external entity communication interface 224 may be supported by one or more radio circuitry. For instance, the external computing entity 112a may include an antenna 226, a transmitter 228 (e.g., radio), and/or a receiver 230 (e.g., radio).

Signals provided to and received from the transmitter 228 and the receiver 230, correspondingly, may include signaling information/data in accordance with air interface standards of applicable wireless systems. In this regard, the external computing entity 112a may be capable of operating with one or more air interface standards, communication protocols, modulation types, and access types. More particularly, the external computing entity 112a may operate in accordance with any of a number of wireless communication standards and protocols, such as those described above with regard to the predictive computing entity 102.

Via these communication standards and protocols, the external computing entity 112a may communicate with various other entities using means such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The external computing entity 112a may also download changes, add-ons, and updates, for instance, to its firmware, software (e.g., including executable instructions, applications, program modules), operating system, and/or the like.

According to one embodiment, the external computing entity 112a may include location determining embodiments, devices, modules, functionalities, and/or the like. For example, the external computing entity 112a may include outdoor positioning embodiments, such as a location module adapted to acquire, for example, latitude, longitude, altitude, geocode, course, direction, heading, speed, universal time (UTC), date, and/or various other information/data. In one embodiment, the location module may acquire data, such as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data may be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data may be determined by triangulating a position of the external computing entity 112a in connection with a variety of other systems, including cellular towers, Wi-Fi access points, and/or the like. Similarly, the external computing entity 112a may include indoor positioning embodiments, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops), and/or the like. For instance, such technologies may include the iBeacons, Gimbal proximity beacons, Bluetooth Low Energy (BLE) transmitters, NFC transmitters, and/or the like. These indoor positioning embodiments may be used in a variety of settings to determine the location of someone or something within inches or centimeters.

The external entity I/O elements 218 may include one or more external entity output devices 220 and/or one or more external entity input devices 222 that may include one or more sensory devices described herein with reference to the I/O elements 114. In some embodiments, the external entity I/O element 218 may include a user interface (e.g., a display, speaker, and/or the like) and/or a user input interface (e.g., keypad, touch screen, microphone, and/or the like) that may be coupled to the external entity processing element 210.

For example, the user interface may be a user application, browser, and/or similar words used herein interchangeably executing on and/or accessible via the external computing entity 112a to interact with and/or cause the display, announcement, and/or the like of information/data to a user. The user input interface may include any of a number of input devices or interfaces allowing the external computing entity 112a to receive data including, as examples, a keypad (hard or soft), a touch display, voice/speech interfaces, motion interfaces, and/or any other input device. In embodiments including a keypad, the keypad may include (or cause display of) the conventional numeric (0-9) and related keys (#, *, and/or the like), and other keys used for operating the external computing entity 112a and may include a full set of alphabetic keys or set of keys that may be activated to provide a full set of alphanumeric keys. In addition to providing input, the user input interface may be used, for example, to activate or deactivate certain functions, such as screen savers, sleep modes, and/or the like.

### III. Examples of Certain Terms

In some embodiments, the term “user” refers to an entity that is associated with a continuous glucose monitor (CGM). For example, a user may wear a CGM for at least an initial time period to generate recorded sensor values for the user. A CGM device, for example, may use a sensor inserted just below the skin to measure glucose levels in a user's interstitial fluid. CGMs generate glucose readings at a predetermined frequency, such as every five minutes, and/or the like. A user may wear a CGM device to manage diabetes by providing real-time knowledge of blood glucose levels and/or the effect the user's behaviors have on their glucose levels. In some examples, the real-time knowledge of blood glucose levels may be facilitated by recorded sensor values.

In some embodiments, the term “recorded sensor value” refers to a data entity that describes a processed or raw glucose reading for a user. A recorded sensor value may be generated by a CGM device for an individual user at a predetermined frequency. A recorded sensor value may include an estimate of glucose concentration in the user's body paired with a timestamp that allows for chronological ordering of a plurality of recorded sensor values and a determination of elapsed time between any two recorded sensor values.

In some embodiments, a recorded sensor value is processed to align and smooth data collected over a time period. For example, a plurality of recorded sensor values may be aligned though one or more data alignment operations. A data alignment operation, for example, may include modifying a timestamp for a recorded sensor value to reassign the timestamp to a nearest five-minute increment on a standard time scale (e.g., timestamps for 9:54 and 9:56 would each be reassigned to time 9:55—the nearest five-minute increment). By aligning the recorded sensor values with a standard time scale, the recorded sensor values may be fit to a uniform framework that allows them to be more easily manipulated and compared.

In some embodiments, a recorded sensor value is further processed to smooth the data collected over a time period. For example, a plurality of recorded sensor values may be smoothened through one or more data smoothing operations. A data smoothing operation, for example, may include generating an average of multiple sensor readings from time increments immediately before and/or after a given time increment. By way of example, a moving average of three sensor readings may be leveraged to generate a recorded sensor value. For example, a recorded sensor value for time 9:55 may include an average of one or more time-aligned glucose readings 9:45, 9:50, and 9:55.

In some embodiments, a plurality of recorded sensor values are recorded for a user during an initial time period to generate one or more predictive insights for the user in accordance with various aspects of the present disclosure.

In some embodiments, the term “initial time period” refers to a period of time in which one or more initial recorded sensor values are recorded for a user. An initial time period may include any time period during which a plurality of recorded sensor values are collected for a user. In some examples, an initial time period may include a 14-day time period. In such a case, the plurality of recorded sensor values may include 14 days' worth of recorded sensor values collected during an initial 14-day time period after a user first begins wearing a CGM device.

In some embodiments, the term “initial physiological feature” refers to a metric for a user that is derived from a plurality of recorded sensor values. In some examples, an initial physiological feature may include one or more metrics that are indicative of (e.g., include identifiers identifying) a daily central tendency for a user over an initial time period. By way of example, an initial physiological feature may be indicative of (e.g., include identifiers identifying) an aggregated glucose value, an aggregated glucose variability value, and/or the like.

In some embodiments, the term “aggregated glucose value” refers to an initial physiological feature that describes a representative glucose level for a user. An aggregated glucose value, for example, may include an aggregated estimated glucose value (EGV) for a user over an initial time period. For example, a daily median EGV may be generated for a user for each day of the initial time period. In some examples, an aggregated glucose value may include an arithmetic average of the daily median EGV's for a user over an initial time period.

In some embodiments, the term “aggregated glucose variability value” refers to an initial physiological feature that describes a variability of a user's glucose levels. An aggregated glucose variability value, for example, may include an aggregated daily glycemic variability for a user over an initial time period. For example, a daily glycemic variability may be generated, using a standard deviation of recorded sensor values, for a user for each day of the initial time period. In some examples, an aggregated glucose variability value may include an arithmetic average of the daily glycemic variability values recorded over the initial time period.

In some embodiments, the term “interaction data object” refers to a data entity that describes a past clinical interaction for a user. A past clinical interaction, for example, may include a medical record, a medical claim, and/or any other recorded medium that is descriptive of a medical event for a user. In some examples, a plurality of interaction data objects may be accessed for a user from electronic health records (EHR) to collect a past medical history of comorbidities and diabetes drug information for the user.

In some embodiments, an interaction data object includes one or more activity codes. An activity code, for example, may include an International Classification of Diseases (ICD) code that is indicative of (e.g., include identifiers identifying) a diagnosis of a disease for a user. In some examples, a plurality of interaction data objects may include a list of ICD codes, such as ICD-10-CM codes, that are recorded for a user during a historical time period. In some examples, each activity code may correspond to an event for a user. The event, for example, may include a diagnosis for a disease corresponding to the activity code. In some examples, the presence of an activity code within a plurality of interaction data objects may be indicative of (e.g., include identifiers identifying) the occurrence of an event (e.g., a disease diagnosis) for a user within a historical time period.

In some embodiments, an activity code is an abstraction from an ICD code. For instance, an activity code may include a chapter code in an ICD taxonomy, such as a chapter in the ICD-10-CM. In some examples, each ICD code within a plurality of interaction data objects for a user may be mapped to a chapter in an ICD-10-CM to provide a high level understanding of the diseases impacting users across different body systems.

In some embodiments, an interaction data object includes data that is indicative of (e.g., include identifiers identifying) a user's insulin usage. An interaction data object, for example, may be indicative of (e.g., include identifiers identifying) an insulin prescription, drug dosage information, and/or the like that identifies an insulin usage pattern for a user.

In some examples, a plurality of interaction data objects may be accessed for a historical time period to identify one or more events and/or insulin usage patterns associated with a user over the historical time period.

In some embodiments, the term “historical time period” refers to a period of time that at least partially precedes an initial time period. A historical time period, for example, may include a time period preceding an initial time period during which a plurality of interaction data objects may be collected to provide contextual information for a user. For example, a plurality of interaction data objects may be collected during a historical time period that precedes the initial time period (e.g, the initial date of CGM wear, etc.). By way of example, the historical time period may include a year preceding the initial date of CGM wear for the user such that if the first day of wearing a CGM device starts on Feb. 1, 2023, the historical time period may span from Feb. 1, 2022 to Feb. 1, 2023.

In some embodiments, the term “activity encoding” refers to an encoded representation for a user that is derived from a plurality of interaction data objects. In some examples, an activity encoding may include one or more encoded representations, such as an event encoding and/or medication usage encoding, that encode robust data insights for a user in a compressed data structure.

In some embodiments, the term “event encoding” refers to an activity encoding that is descriptive of a plurality of events for a user during a historical time period. An event encoding may include an encoded data representation, such as a one-hot encoding, an embedding, and/or the like, that represents robust event history in a compressed data structure.

In some embodiments, an event encoding is a one-hot encoding indicative of (e.g., include identifiers identifying) the presence of one or more activity codes within a plurality of interaction data objects. By way of example, a first binary value (e.g., 1, etc.) may be assigned if a user is associated with a particular activity code. Otherwise, a second binary value (e.g., 0, etc.) may be assigned.

In some embodiments, an event encoding is a feature embedding indicative of (e.g., include identifiers identifying) the presence of one or more activity codes within a plurality of interaction data objects. A feature embedding, for example, may be generated by a previously trained machine learning model (e.g., ICD2Vec, etc.) to output the feature embedding.

In some embodiments, the term “medication usage encoding” refers to an activity encoding that is descriptive of an insulin usage pattern for a user during a historical time period. A medication usage encoding may include an encoded data representation, such as a one-hot encoding, an embedding, and/or the like, that represents robust medication usage history, such as insulin, other diabetes medication classes, and/or the like in a compressed data structure.

In some embodiments, a medication usage encoding is a one-hot encoding indicative of (e.g., include identifiers identifying) the usage of insulin for a user. By way of example, a first binary value (e.g., 1, etc.) may be assigned if a user has taken insulin (e.g., during a day, etc.) during a historical time period. Otherwise, a second binary value (e.g., 0, etc.) may be assigned. In addition, or alternatively, a medication usage encoding is a feature embedding indicative of (e.g., include identifiers identifying) a drug usage and/or dosage prescribed within a plurality of interaction data objects.

In some embodiments, the term “combined input feature vector” refers to an input to a predictive machine learning model. A combined input feature vector may be tailored to a predictive machine learning model. In some examples, a combined input feature vector may include an aggregation of the initial physiological features and/or activity encodings for a user. By way of example, a combined input feature vector may include the aggregated glucose value, the aggregated glucose variability value, the event encoding, and/or the medication usage encoding for the user. In addition, or alternatively, a combined input feature vector may include a feature embedding for the user. The feature embedding may be generated by a predictive machine learning model based on the aggregated glucose value, the aggregated glucose variability value, the event encoding, and/or the medication usage encoding for the user.

In some embodiments, the term “machine learning model” refers to a data entity that describes parameters, hyper-parameters, and/or defined operations of a rules-based and/or machine learning model (e.g., model including at least one of one or more rule-based layers, one or more layers that depend on trained parameters, coefficients, and/or the like). The predictive machine learning model may include any type of model configured, trained, and/or the like to generate a physiological prediction based on a combined input feature vector for a user. The predictive machine learning model may include one or more of any type of machine learning model including one or more supervised, unsupervised, semi-supervised, reinforcement learning models, and/or the like. For instance, the predictive machine learning model may include a supervised model that may be trained using a labeled training dataset. In some examples, the predictive machine learning model may include multiple models configured to perform one or more different stages of a prediction process. The models, for example, may include a linear regression model, a neural network, a random forest model, and/or the like.

In some embodiments, the term “physiological prediction” refers to an output of a predictive machine learning model. A physiological prediction may be a predicted average glucose metric for a user at a future time period.

In some embodiments, the term “future time period” refers to a period of time that is at least partially subsequent to an initial time period. A future time period may correspond to a physiological prediction for a user. For example, a future time period may include a 90-day time period (e.g., 75 days in advance of an initial time period). In some examples, a physiological prediction may include a 90-day average glucose metric for a user.

In some embodiments, the term “prediction-based action” describes an output for a user based on a physiological prediction. A prediction-based action may include one or more computing actions, such as issuing one or more alerts or notifications, generating and providing one or more treatment recommendations, generating and rendering one or more instructions, generating and/or providing one or more computing instructions, and/or the like. In some examples, in a clinical domain, a prediction-based action may include a clinical plan recommendation based on the physiological prediction for the user.

In some embodiments, the term “labeled training dataset” refers to a dataset with one or more training input data objects and/or one or more training labels corresponding to the training input data objects. Each training input data object, for example, may include a historical and/or synthetic entity that represents a combined input feature vector for a predictive machine learning model. A training input data object may correspond with a training label that describes a target output for the predictive machine learning model, such as a physiological prediction. By way of example, a training input data object may include a historical recorded sensor value and a training label may include a historical combined input feature vector. A historical recorded sensor value, for example, may include previously recorded sensor values for a user. A historical combined input feature vector may include combined input feature vectors that are generated for the user based on data that precedes a historical recorded sensor value by a target time period. In some examples, a historical recorded sensor value may be a ground truth for a historical combined input feature vector.

### IV. Overview

Embodiments of the present disclosure present new signal interpretation techniques to improve computer interpretation of physiological signals for a user. To do so, the present disclosure describes a predictive machine learning model that is trained to interpret a combined input feature vector storing an aggregation of limited physiological signals contextualized with historical user information. The predictive machine learning model includes a new machine-learning model that is trained using a labeled training dataset directly tailored for a new input, the combined input feature vector, to improve the accuracy and range of predictions for a user. Once trained, a combined input feature vector may be generated for a user in real time and input to the machine learning model to receive real time and future predictive insights for the user. In this way, some techniques of the present disclosure enable the generation of predictive insights with increased range and accuracy compared with traditional techniques, while consuming less data and processing resources.

When applied to clinical environments, some techniques of the present disclosure allow for more rapid identification of effective diabetic treatment and identification of users whose symptoms are harder to manage. In particular, some techniques of the present disclosure may generate physiological predictions that may reduce costs incurred from ineffective treatment options by identifying optimal treatments using initial sensor values over a short initial time period. Moreover, some techniques of the present disclosure may reduce costs from health complications due to ineffective treatment and identify cohorts of individuals who may not respond effectively to certain treatment/treatments. Unlike traditional techniques for interpreting physiological signals, some embodiments of the present disclosure deliver robust results even with lower frequency of sensor information, such as CGM data. The ability to perform with low frequency CGM data results in substantial cost savings by reducing the time required for each user to wear a CGM device and avoiding costly versions of CGM that provide higher frequency data. Ultimately, these improvements may lead to improved health outcomes of cohorts of users and reduced claim costs.

Some technologically advantageous embodiments of the present disclosure include (i) techniques for generating a new input vector to improve the predictive performance of a machine learning model, (ii) techniques for using and training a machine learning model for generating physiological prediction for a user with limited physiological information, and (iii) prediction techniques that leverage initial physiological sensor values to predict future physiological sensor values for a user among other aspects of the present disclosure. Other technical improvements and advantages may be realized by one of ordinary skill in the art.

### V. Example System Operations

As described below, various embodiments of the present disclosure leverage new input data structures and machine learning techniques to make important technical contributions to the interpretation of physiological signals recorded by sensors. In this manner, some embodiments described here may leverage combined feature vectors to generate and then leverage a machine learning model to improve upon traditional predictive insights for a user.

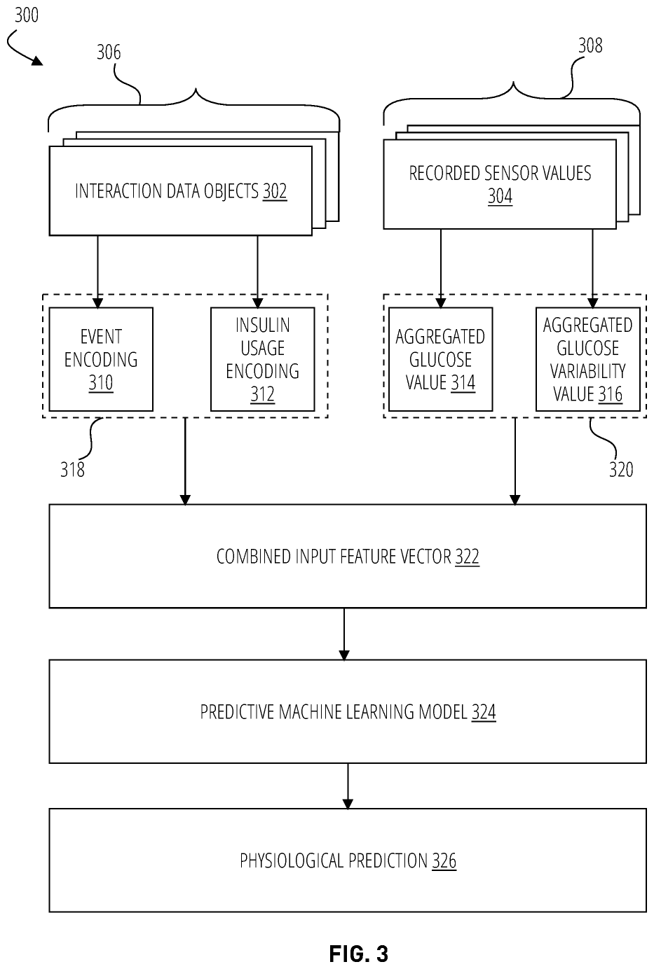

FIG. 3 is a dataflow diagram 300 showing example data structures and modules for interpreting short term physiological signals in accordance with some embodiments discussed herein. The dataflow diagram 300 depicts a signal interpretation technique for improving the interpretation of recorded signals for a user. The signal interpretation technique leverages a new data structure, the combined input feature vector 322, that combines interaction data objects 302 and recorded sensor values 304 from a user into a machine readable data structure. The combined input feature vector 322 may be input to a predictive machine learning model 324 that is specifically configured to generate a physiological prediction 326 for the user based on the combined input feature vector 322.

In some embodiments, a user is an entity that is associated with a CGM device. For example, a user may wear a CGM for at least an initial time period 308 to generate recorded sensor values for the user. A CGM device, for example, may use a sensor inserted just below the skin to measure glucose levels in a user's interstitial fluid. CGMs generate glucose readings at a predetermined frequency, such as every five minutes, an hour, and/or the like. A user may wear a CGM device to manage diabetes by providing real-time knowledge of blood glucose levels and/or the effect the user's behaviors have on their glucose levels. In some examples, the real-time knowledge of blood glucose levels may be facilitated by the recorded sensor values 304.

In some embodiments, the recorded sensor values 304 are data entities that describe a processed or raw glucose reading for a user. The recorded sensor values 304 may be generated by a CGM device for an individual user at a predetermined frequency. The recorded sensor values 304 may include an estimate of glucose concentration in the user's body paired with a timestamp that allows for chronological ordering of the recorded sensor values 304 and a determination of elapsed time between any two recorded sensor values 304.

In some embodiments, the recorded sensor values 304 are processed to align and smooth data collected over a time period, such as the initial time period 308. For example, the plurality of recorded sensor values 304 may be aligned though one or more data alignment operations. A data alignment operation, for example, may include modifying a timestamp for a recorded sensor value to reassign the timestamp to a nearest five-minute increment on a standard time scale (e.g., timestamps for 9:54 and 9:56 would each be reassigned to time 9:55—the nearest five-minute increment). By aligning the recorded sensor values 304 with a standard time scale, the recorded sensor values 304 may be fit to a uniform framework that allows for more accurate data processing and interpretation.

In some embodiments, the recorded sensor values 304 are further processed to smooth the data collected over a time period, such as the initial time period 308. For example, the plurality of recorded sensor values 304 may be smoothened through one or more data smoothing operations. A data smoothing operation, for example, may include generating an average of multiple sensor readings from time increments immediately before and/or after a given time increment. By way of example, a moving average of three sensor readings may be leveraged to generate a recorded sensor value. For example, a recorded sensor value for time 9:55 may include an average of one or more time-aligned sensor readings 9:45, 9:50, and 9:55.

In some embodiments, a plurality of recorded sensor values 304 are recorded for a user during the initial time period 308 to generate one or more predictive insights for the user in accordance with various aspects of the present disclosure. In some embodiments, the initial time period 308 is a period of time in which one or more initial recorded sensor values 304 are recorded for the user. The initial time period 308 may include any time period during which a plurality of recorded sensor values 304 are collected for the user. In some examples, the initial time period 308 may include a 14-day time period. In such a case, the plurality of recorded sensor values 304 may include 14 days' worth of recorded sensor values 304 collected during an initial 14-day time period after a user first begins wearing a CGM device.

In some embodiments, one or more initial physiological features 320 are received (and/or generated) for the user that are based on a plurality of recorded sensor values 304 for the user. In some examples, the one or more initial physiological features 320 may include an aggregated glucose value 314 and/or an aggregated glucose variability value 316. The aggregated glucose value 314 may include an arithmetic average of a plurality of daily median recorded sensor measurements for the user over the initial time period 308. The aggregated glucose variability value 316 may include an arithmetic average of a plurality of daily median glycemic variability measurements for the user over the initial time period 308.

In some embodiments, the one or more initial physiological features 320 are metrics for a user that are derived from a plurality of recorded sensor values 304. In some examples, the initial physiological features 320 may include one or more metrics that are indicative of a daily central tendency for a user over the initial time period 308. By way of example, the initial physiological features 320 may be indicative of an aggregated glucose value 314, an aggregated glucose variability value 316, and/or the like.

In some embodiments, the aggregated glucose values 314 are initial physiological features 320 that describe a representative glucose level for a user. An aggregated glucose value 314, for example, may include an aggregated estimated glucose value (EGV) for a user over the initial time period 308. For example, a daily median EGV may be generated for a user for each day of the initial time period 308. In some examples, the aggregated glucose value 314 may include an arithmetic average of the daily median EGV's for a user over the initial time period 308.

In some embodiments, the aggregated glucose variability values 316 are initial physiological features 320 that describe a variability of a user's glucose levels. An aggregated glucose variability value 316, for example, may include an aggregated daily glycemic variability for a user over the initial time period 308. For example, a daily glycemic variability may be generated, using a standard deviation of the recorded sensor values 304, for a user for each day of the initial time period 308. In some examples, the aggregated glucose variability value 316 may include an arithmetic average of the daily glycemic variability values recorded over the initial time period 308.

In some embodiments, a combined input feature vector 322 is generated for the user by aggregating the one or more initial physiological features 320 with one or more activity encodings 318. For example, one or more activity encodings 318 may be generated for the user based on a plurality of interaction data objects 302 for the user.

In some embodiments, the plurality of interaction data objects 302 are data entities that describe a past clinical interaction for the user. A past clinical interaction, for example, may include a medical record, a medical claim, and/or any other recorded medium that is descriptive of a medical event for the user. In some examples, a plurality of interaction data objects 302 may be accessed for the user from electronic health records (EHRs) to collect a past medical history of comorbidities and diabetes drug information for the user.

In some embodiments, each of the interaction data objects 302 include one or more activity codes. An activity code, for example, may include an International Classification of Diseases (ICD) code that is indicative of a diagnosis of a disease for a user. In some examples, a plurality of interaction data objects 302 may include a list of ICD codes, such as ICD-10-CM codes, that are recorded for the user during a historical time period 306. In some examples, each activity code may correspond to an event for a user. The event, for example, may include a diagnosis for a disease corresponding to the activity code. In some examples, the presence of an activity code within a plurality of interaction data objects 302 may be indicative of the occurrence of an event (e.g., a disease diagnosis) for a user within the historical time period 306.

In some embodiments, an activity code is an abstraction from an ICD code. For instance, an activity code may include a chapter code in an ICD taxonomy, such as a chapter in the ICD-10-CM. In some examples, each ICD code within a plurality of interaction data objects 302 for a user may be mapped to a chapter in an ICD-10-CM to provide a high level understanding of the diseases impacting users across different body systems.

In some embodiments, the interaction data objects 302 include data that is indicative of a user's insulin usage. The interaction data objects 302, for example, may be indicative of an insulin prescription, drug dosage information, and/or the like that identifies an insulin usage pattern for a user during the historical time period 306.

In some examples, a plurality of interaction data objects 302 may be accessed for the historical time period 306 to identify one or more events and/or insulin usage patterns associated with the user over the historical time period 306.

In some embodiments, the historical time period 306 is a period of time that at least partially precedes the initial time period 308. The historical time period 306, for example, may include a time period preceding the initial time period 308 during which a plurality of interaction data objects 302 may be collected to provide contextual information for the user. For example, a plurality of interaction data objects 302 may be collected during the historical time period 306 that precedes the initial time period 308 (e.g, the initial date of CGM wear, etc.). By way of example, the historical time period 306 may include a year preceding the initial date of CGM wear for the user such that if the first day of wearing a CGM device starts on Feb. 1, 2023, the historical time period 306 may span from Feb. 1, 2022 to Feb. 1, 2023.

In some embodiments, the one or more activity encodings include an event encoding 310 and/or a medication usage encoding 312. In some examples, the event encoding 310 may include a first one-hot encoding indicative of a presence or an absence of one or more events for a user during the historical time period 306 preceding the initial time period 308. For example, each of the plurality of interaction data objects 302 may include one or more activity codes and the presence and/or the absence of the one or more events may be based on the one or more activity codes. In some examples, the medication usage encoding 312 may include a second one-hot encoding indicative of an insulin usage pattern for the user during the historical time period 306. For example, the insulin usage pattern may be indicative of a daily usage pattern of the user and the medication usage encoding 312 may be indicative of a daily binary indicator indicative of a use and/or nonuse of insulin each day of the historical time period 306.

In some embodiments, the activity encodings 318 are encoded representations for the user that are derived from a plurality of interaction data objects 302. In some examples, an activity encoding may include one or more encoded representations, such as an event encoding 310 and/or medication usage encoding 312, that encode robust data insights for a user in a compressed data structure.

In some embodiments, the event encoding 310 is an activity encoding 318 that is descriptive of a plurality of events for a user during the historical time period 306. The event encoding 310 may include an encoded data representation, such as a one-hot encoding, an embedding, and/or the like, that represents robust event history in a compressed data structure.

In some embodiments, the event encoding 310 is a one-hot encoding indicative of the presence of one or more activity codes within the plurality of interaction data objects 302. By way of example, a first binary value (e.g., 1, etc.) may be assigned if a user is associated with a particular activity code. Otherwise, a second binary value (e.g., 0, etc.) may be assigned.

In some embodiments, the event encoding 310 is a feature embedding indicative of the presence of one or more activity codes within the plurality of interaction data objects 302. A feature embedding, for example, may be generated by a machine learning model (e.g., ICD2Vec, etc.) previously trained to output the feature embedding.

In some embodiments, the medication usage encoding 312 is an activity encoding 318 that is descriptive of an insulin usage pattern for a user during the historical time period 306. The medication usage encoding 312 may include an encoded data representation, such as a one-hot encoding, an embedding, and/or the like, that represents robust insulin usage history in a compressed data structure.

In some embodiments, the medication usage encoding 312 is a one-hot encoding indicative of the usage of insulin for a user. By way of example, a first binary value (e.g., 1, etc.) may be assigned if a user has taken insulin (e.g., during a day, etc.) during the historical time period 306. Otherwise, a second binary value (e.g., 0, etc.) may be assigned. In addition, or alternatively, the medication usage encoding 312 may be a feature embedding indicative of a drug usage and/or dosage prescribed within the plurality of interaction data objects 302.

In some embodiments, a physiological prediction 326 is generated for the user, using the predictive machine learning model 324, based on the combined input feature vector 322. In some examples, the physiological prediction 326 for the user may include a plurality of predicted average sensor values for the user during a future time period subsequent to the initial time period 308. The future time period, for example, may include a seventy five day time period and the plurality of predicted average sensor values may include a predicted daily average sensor value for one or more days of the seventy five day time period.

In some embodiments, the predictive machine learning model 324 is a data entity that describes parameters, hyper-parameters, and/or defined operations of a rules-based and/or machine learning model (e.g., model including at least one of one or more rule-based layers, one or more layers that depend on trained parameters, coefficients, and/or the like). The predictive machine learning model 324 may include any type of model configured, trained, and/or the like to generate a physiological prediction 326 based on the combined input feature vector 322 for a user. The predictive machine learning model 324 may include one or more of any type of machine learning model including one or more supervised, unsupervised, semi-supervised, reinforcement learning models, and/or the like. For instance, the predictive machine learning model 324 may include a supervised model that may be trained using a labeled training dataset. In some examples, the predictive machine learning model 324 may include multiple models configured to perform one or more different stages of a prediction process. The models, for example, may include a linear regression model, a neural network, a random forest model, and/or the like.

In some embodiments, the combined input feature vector 322 is an input to the predictive machine learning model 324. The combined input feature vector 322 may be tailored to the predictive machine learning model 324. In some examples, the combined input feature vector 322 may include an aggregation of the initial physiological features 320 and/or activity encodings 318 for the user. By way of example, the combined input feature vector 322 may include the aggregated glucose value 314, the aggregated glucose variability value 316, the event encoding 310, and/or the medication usage encoding 312 for the user. In addition, or alternatively, the combined input feature vector 322 may include a feature embedding for the user. The feature embedding may be generated by the predictive machine learning model 324 based on the aggregated glucose value 314, the aggregated glucose variability value 316, the event encoding 310, and/or the medication usage encoding 312 for the user.

In some embodiments, the physiological prediction 326 is an output of the predictive machine learning model 324. The physiological prediction may be a predicted average glucose metric for a user at a future time period. In some embodiments, the future time period is a period of time that is at least partially subsequent to the initial time period 308. The future time period may correspond to a physiological prediction 326 for the user. For example, the future time period may include a 90-day time period (e.g., 75 days in advance of an initial time period). In some examples, the physiological prediction 326 may include a 90-day average glucose metric for the user.

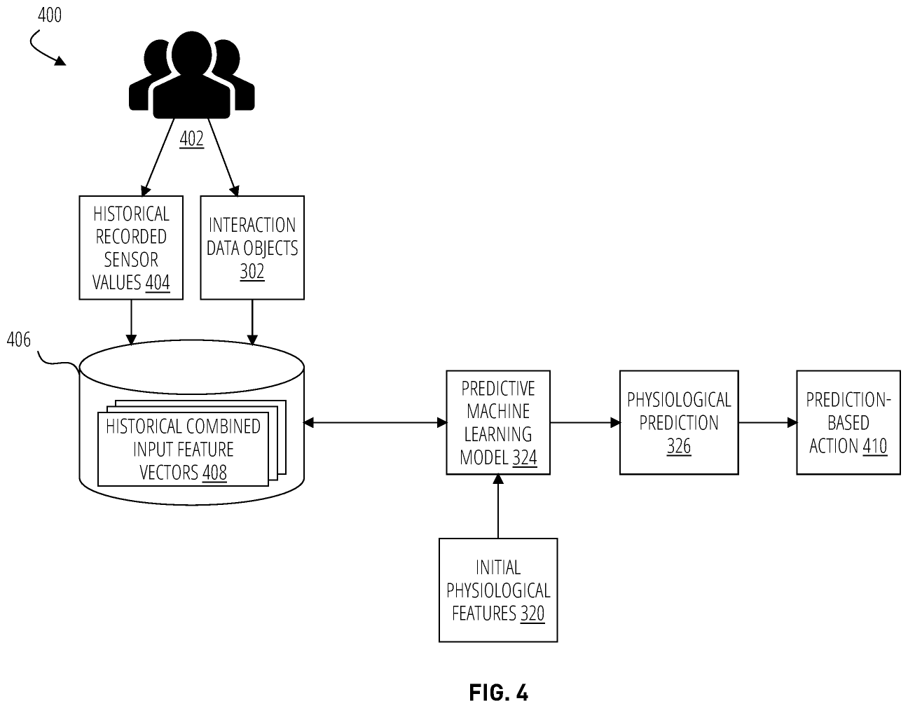

In some embodiments, the predictive machine learning model 324 is previously trained on a labeled training dataset that includes a plurality of historical combined input feature vectors and a plurality of corresponding historical recorded sensor values. In some examples, the labeled training dataset may be updated with the combined input feature vector 322 and a plurality of corresponding future recorded sensor values for the user. An example of a process for training the predictive machine learning model 324 will now further be described with reference to FIG. 4.

FIG. 4 is a dataflow diagram 400 showing example data structures and modules for training a machine learning model to interpret short term physiological signals in accordance with some embodiments discussed herein. The dataflow diagram 400 depicts new training techniques that leverage historical real world data to generate training data structures for refining the predictive machine learning model 324. The real world data, for example, may include historical recorded sensor values 404 and interaction data objects 302 for a cohort 402 of users. The historical recorded sensor values 404 and interaction data objects 302, for example, may include interaction data objects 302 and historical recorded sensor values 404 for a user and/or one or more additional. For example, the data may be aggregated from a cohort 402 of users to generate a labeled training dataset 406 for training the predictive machine learning model 324.

In some embodiments, the labeled training dataset 406 includes a dataset with one or more training input data objects and/or one or more training labels corresponding to the training input data objects. Each training input data object, for example, may include a historical and/or synthetic entity that represents a combined input feature vector for the predictive machine learning model 324. A training input data object may correspond with a training label that describes a target output for the predictive machine learning model 324, such as a physiological prediction 326. By way of example, a training input data object may include a historical recorded sensor value 404 and a training label may include a historical combined input feature vector 408. The historical recorded sensor value 404, for example, may include previously recorded sensor values for a user. A historical combined input feature vector 408 may include combined input feature vectors that are generated for the user based on data that precedes the historical recorded sensor value 404 by a target time period (e.g., 75 days, 90 days, etc.). In some examples, a historical recorded sensor value 404 may be a ground truth for a historical combined input feature vector 408.

In some embodiments, the predictive machine learning model 324 is trained, using one or more supervisory training techniques, such as back propagation of errors, using the labeled training dataset 406. Once trained, the predictive machine learning model 324 may receive initial physiological features 320 from a user. The initial physiological features 320 may be combined with data derived from interaction data objects 302 for the user, using some of the techniques described herein, to generate a combined input feature vector for the user. In some examples, the interaction data objects 302 may also be included in the labeled training dataset 406. The combined input feature vector may be provided as input to the trained predictive machine learning model 324 and, in response to the combined input feature vector, the predictive machine learning model 324 may output a physiological prediction 326 for the user. In some examples, the performance of a prediction-based action 410 may be initiated based on the physiological prediction 326.

In some embodiments, a prediction-based action 410 describes an output for a user based on the physiological prediction 326. The prediction-based action 410, for example, may include one or more computing actions, such as issuing one or more alerts or notifications, generating and providing one or more treatment recommendations, generating and rendering one or more instructions, generating and/or providing one or more computing instructions, and/or the like. In some examples, in a clinical domain, the prediction-based action 410 may include a clinical plan recommendation based on the physiological prediction 326 for the user.

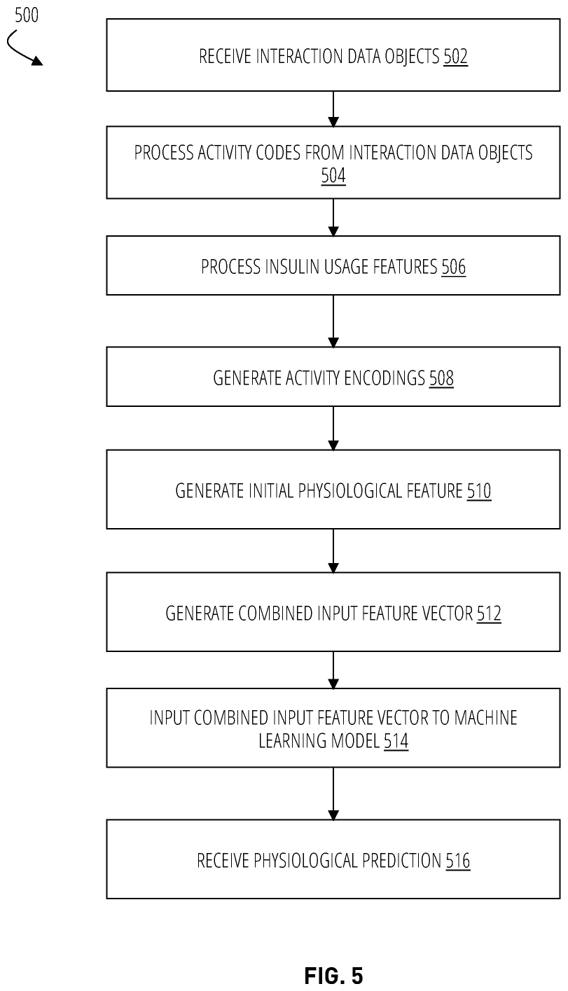

FIG. 5 is a flowchart showing an example of a process 500 for interpreting short term physiological signals in accordance with some embodiments discussed herein. The flowchart depicts a signal interpretation process for generating a predictive output from a combination of physiological and historical insights for a user to overcome various limitations of traditional signal interpretation processes that lack accuracy and rely on robust, hard to manage datasets. The signal interpretation process may be implemented by one or more computing devices, entities, and/or systems described herein. For example, via the various steps/operations of the process 500, the computing system 100 may generate a physiological prediction that accurately represents a user's physiological signals at a future time period that extends beyond time periods previously achieved using traditional methods. Moreover, unlike traditional signal interpretation techniques, the process 500 creates a condensed data structure for processing by a machine learning model that encodes both current physiological signals and historical contextual information. By grounding predictive insights on information stored within a condensed data structure, the process 500 enables the generation of robust labeled training datasets that may be updated over time to tailor a model to a user, a population of users, and/or trends within a user population.

FIG. 5 illustrates an example process 500 for explanatory purposes. Although the example process 500 depicts a particular sequence of steps/operations, the sequence may be altered without departing from the scope of the present disclosure. For example, some of the steps/operations depicted may be performed in parallel or in a different sequence that does not materially impact the function of the process 500. In other examples, different components of an example device or system that implements the process 500 may perform functions at substantially the same time or in a specific sequence.

In some embodiments, the process 500 includes, at step/operation 502, receiving interaction data objects for a user. For example, the computing system 100 may receive the interaction data objects. In some examples, each of the plurality of interaction data objects may include one or more activity codes.

In some embodiments, the process 500 includes, at step/operation 504, processing activity codes from the interaction data objects. For example, the computing system 100 may process the activity codes from the interaction data objects.

In some embodiments, the process 500 includes, at step/operation 506, processing medication usage features from the interaction data objects. For example, the computing system 100 may process the medication usage features from the interaction data objects.

In some embodiments, the process 500 includes, at step/operation 508, generating activity encodings for the user. For example, the computing system 100 may generate the activity encodings for the user. For instance, the computing system 100 may generate one or more activity encodings for the user based on the plurality of interaction data objects for the user. The one or more activity encodings may include an event encoding and/or a medication usage encoding. The event encoding may be a first one-hot encoding indicative of a presence or an absence of one or more events for a user during a historical time period preceding an initial time period. In some examples, the presence and/or the absence of the one or more events may be based on the one or more activity codes within the interaction data objects. The medication usage encoding may include a second one-hot encoding indicative of an insulin usage pattern for the user during the historical time period. In some examples, the insulin usage pattern may be indicative of a daily usage pattern of the user and the medication usage encoding may include a daily binary indicator indicative of a use and/or nonuse of insulin each day of the historical time period.

In some embodiments, the process 500 includes, at step/operation 510, generating initial physiological features for the user. For example, the computing system 100 may generate the initial physiological features for the user. For instance, the computing system 100 may receive one or more initial physiological features for a user that are based on a plurality of recorded sensor values for the user. The one or more initial physiological features may include an aggregated glucose value and/or an aggregated glucose variability value. The aggregated glucose value may include an arithmetic average of a plurality of daily median recorded sensor measurements for the user over an initial time period. The aggregated glucose variability value may include an arithmetic average of a plurality of daily median glycemic variability measurements for the user over the initial time period.

In some embodiments, the process 500 includes, at step/operation 512, generating a combined input feature vector for the user. For example, the computing system 100 may generate the combined input feature vector for the user. For instance, the computing system 100 may generate a combined input feature vector for the user by aggregating the one or more initial physiological features and the one or more activity encodings. In this manner, one condensed data structure may be generated that represents both physiological and contextual data for a user. These different types of data may be leveraged together, by a machine learning model, to generate improved predictions that are not achievable by traditional signal processing techniques.

In some embodiments, the process 500 includes, at step/operation 514, inputting the combined input feature vector to a machine learning model. For example, the computing system 100 may input the combined input feature vector to a predictive machine learning model.

In some embodiments, the process 500 includes, at step/operation 516, receiving a physiological prediction for the user. For example, the computing system 100 may receive the physiological prediction. For instance, the computing system 100 may generate, using the machine learning model, the physiological prediction for the user based on the combined input feature vector. In some examples, the physiological prediction for the user may include a plurality of predicted average sensor values for the user during a future time period subsequent to the initial time period. The future time period may include a seventy five day time period and the plurality of predicted average sensor values may include a predicted daily average sensor value for one or more days of the seventy five day time period.

In some examples, the machine learning model is previously trained on a labeled training dataset that includes a plurality of historical combined input feature vectors and a plurality of corresponding historical recorded sensor values. In some examples, the labeled training dataset may be updated with the combined input feature vector and a plurality of corresponding future recorded sensor values for the user. In this manner, the machine learning model may be configured to anticipate and adapt to historical trends over time.

In some embodiments, the process 500 includes initiating the performance of a prediction-based action based on physiological prediction. For example, some techniques of the present disclosure enable the generation of action outputs that may be performed to initiate one or more prediction-based actions to achieve real-world effects. The signal interpretation techniques of the present disclosure may be used, applied, and/or otherwise leveraged to generate a machine learning model and physiological predictions output therefrom, which may help in the signal interpretation for a user. The machine learning model of the present disclosure may be leveraged to initiate the performance of various computing tasks that improve the performance of a computing system (e.g., a computer itself, etc.) with respect to various prediction-based actions performed by the computing system 100. Example prediction-based actions may include the generation of alerts and/or actions to automatically address physiological predictions for a user.

In some examples, the computing tasks may include prediction-based actions that may be based on a prediction domain. A prediction domain may include any environment in which computing systems may be applied to achieve real-word insights, such as predictions (e.g., physiological predictions, etc.), and initiate the performance of computing tasks, such as prediction-based actions (e.g., alerting a user, prompting behavior for improving predictions, generating a health alert, etc.) to act on the real-world insights. These prediction-based actions may cause real-world changes, for example, by controlling a hardware component, providing alerts, interactive actions, and/or the like.

Examples of prediction domains may include financial systems, clinical systems, autonomous systems, robotic systems, and/or the like. Prediction-based actions in such domains may include the initiation of automated instructions across and between devices, automated notifications, automated scheduling operations, automated precautionary actions, automated security actions, automated data processing actions, automated data compliance actions, automated data access enforcement actions, automated adjustments to computing and/or human data access management, and/or the like.

In some embodiments, the signal interpretation techniques of the process 500 are applied to initiate the performance of one or more prediction-based actions. A prediction-based action may depend on the prediction domain. In some examples, the computing system 100 may leverage the signal interpretation techniques to initiate a medication change, new behavior recommendations, user alerts, and/or any other operation for handling physiological predictions for a user.

### VI. Conclusion

Many modifications and other embodiments will come to mind to one skilled in the art to which the present disclosure pertains having the benefit of the teachings presented in the foregoing descriptions and the associated drawings. Therefore, it is to be understood that the present disclosure is not to be limited to the specific embodiments disclosed and that modifications and other embodiments are intended to be included within the scope of the appended claims. Although specific terms are employed herein, they are used in a generic and descriptive sense only and not for purposes of limitation.

### VII. Examples

Example 1. A computer-implemented method, the computer-implemented method comprising receiving, by one or more processors, one or more initial physiological features for a user that are based on a plurality of recorded sensor values for the user; generating, by the one or more processors, one or more activity encodings for the user based on a plurality of interaction data objects for the user; generating, by the one or more processors, a combined input feature vector for the user by aggregating the one or more initial physiological features and the one or more activity encodings; generating, by the one or more processors and using a machine learning model, a physiological prediction for the user based on the combined input feature vector; and initiating, by the one or more processors, the performance of a prediction-based action based on the physiological prediction.

Example 2. The computer-implemented method of example 1, wherein the one or more initial physiological features comprise an aggregated glucose value and an aggregated glucose variability value.

Example 3. The computer-implemented method of example 2, wherein the aggregated glucose value comprises an arithmetic average of a plurality of daily median recorded sensor measurements for the user over an initial time period.

Example 4. The computer-implemented method of any of examples 2 or 3, wherein the aggregated glucose variability value comprises an arithmetic average of a plurality of daily median glycemic variability measurements for the user over an initial time period.

Example 5. The computer-implemented method of any of the preceding examples, wherein the one or more activity encodings comprise (i) an event encoding comprising a first one-hot encoding indicative of a presence or an absence of one or more events for the user during a historical time period preceding an initial time period corresponding to the plurality of recorded sensor values, and (ii) a medication usage encoding comprising a second one-hot encoding indicative of an insulin usage pattern for the user during the historical time period.

Example 6. The computer-implemented method of example 5, wherein each of the plurality of interaction data objects comprises one or more activity codes and the presence or the absence of the one or more events is based on the one or more activity codes.

Example 7. The computer-implemented method of any of examples 5 or 6, wherein the insulin usage pattern is indicative of a daily usage pattern of the user and the medication usage encoding comprising a daily binary indicator indicative of a use or a nonuse of insulin each day of the historical time period.

Example 8. The computer-implemented method of any of the preceding examples, wherein the physiological prediction for the user comprises a plurality of predicted average sensor values for the user during a future time period subsequent to an initial time period corresponding to the plurality of recorded sensor values.

Example 9. The computer-implemented method of example 8, wherein the future time period comprises a seventy five day time period and the plurality of predicted average sensor values comprises a predicted daily average sensor value for one or more days of the seventy five day time period.

Example 10. The computer-implemented method of any of the preceding examples, wherein the machine learning model is previously trained on a labeled training dataset comprising a plurality of historical combined input feature vectors and a plurality of corresponding historical recorded sensor values.

Example 11. The computer-implemented method of example 10, wherein the labeled training dataset is updated with the combined input feature vector and a plurality of corresponding future recorded sensor values for the user.

Example 12. A computing system comprising memory and one or more processors communicatively coupled to the memory, the one or more processors configured to receive one or more initial physiological features for a user that are based on a plurality of recorded sensor values for the user; generate one or more activity encodings for the user based on a plurality of interaction data objects for the user; generate a combined input feature vector for the user by aggregating the one or more initial physiological features and the one or more activity encodings; generate, using a machine learning model, a physiological prediction for the user based on the combined input feature vector; and initiate the performance of a prediction-based action based on the physiological prediction.

Example 13. The computing system of example 12, wherein the one or more initial physiological features comprise an aggregated glucose value and an aggregated glucose variability value.

Example 14. The computing system of example 13, wherein the aggregated glucose value comprises an arithmetic average of a plurality of daily median recorded sensor measurements for the user over an initial time period.

Example 15. The computing system of any of examples 13 or 14, wherein the aggregated glucose variability value comprises an arithmetic average of a plurality of daily median glycemic variability measurements for the user over an initial time period.

Example 16. The computing system of any of examples 12 through 15, wherein the one or more activity encodings comprise (i) an event encoding comprising a first one-hot encoding indicative of a presence or an absence of one or more events for the user during a historical time period preceding an initial time period corresponding to the plurality of recorded sensor values, and (ii) a medication usage encoding comprising a second one-hot encoding indicative of an insulin usage pattern for the user during the historical time period.

Example 17. The computing system of example 16, wherein each of the plurality of interaction data objects comprises one or more activity codes and the presence or the absence of the one or more events is based on the one or more activity codes.

Example 18. One or more non-transitory computer-readable storage media including instructions that, when executed by one or more processors, cause the one or more processors to receive one or more initial physiological features for a user that are based on a plurality of recorded sensor values for the user; generate one or more activity encodings for the user based on a plurality of interaction data objects for the user; generate a combined input feature vector for the user by aggregating the one or more initial physiological features and the one or more activity encodings; generate, using a machine learning model, a physiological prediction for the user based on the combined input feature vector; and initiate the performance of a prediction-based action based on the physiological prediction.

Example 19. The one or more non-transitory computer-readable storage media of example 18, wherein the physiological prediction for the user comprises a plurality of predicted average sensor values for the user during a future time period subsequent to an initial time period corresponding to the plurality of recorded sensor values.

Example 20. The one or more non-transitory computer-readable storage media of example 19, wherein the future time period comprises a seventy five day time period and the plurality of predicted average sensor values comprises a predicted daily average sensor value for one or more days of the seventy five day time period.

1. A computer-implemented method, the computer-implemented method comprising:
receiving, by one or more processors, one or more initial physiological features for a user that are based on a plurality of recorded sensor values for the user;
generating, by the one or more processors, one or more activity encodings for the user based on a plurality of interaction data objects for the user;
generating, by the one or more processors, a combined input feature vector for the user by aggregating the one or more initial physiological features and the one or more activity encodings;
generating, by the one or more processors and using a machine learning model, a physiological prediction for the user based on the combined input feature vector; and
initiating, by the one or more processors, the performance of a prediction-based action based on the physiological prediction.

2. The computer-implemented method of claim 1, wherein the one or more initial physiological features comprise an aggregated glucose value and an aggregated glucose variability value.

3. The computer-implemented method of claim 2, wherein the aggregated glucose value comprises an arithmetic average of a plurality of daily median recorded sensor measurements for the user over an initial time period.

4. The computer-implemented method of claim 2, wherein the aggregated glucose variability value comprises an arithmetic average of a plurality of daily median glycemic variability measurements for the user over an initial time period.

5. The computer-implemented method of claim 1, wherein the one or more activity encodings comprise:
(i) an event encoding comprising a first one-hot encoding indicative of a presence or an absence of one or more events for the user during a historical time period preceding an initial time period corresponding to the plurality of recorded sensor values, and
(ii) a medication usage encoding comprising a second one-hot encoding indicative of an insulin usage pattern for the user during the historical time period.

6. The computer-implemented method of claim 5, wherein each of the plurality of interaction data objects comprises one or more activity codes and the presence or the absence of the one or more events is based on the one or more activity codes.

7. The computer-implemented method of claim 5, wherein the insulin usage pattern is indicative of a daily usage pattern of the user and the medication usage encoding comprising a daily binary indicator indicative of a use or a nonuse of insulin each day of the historical time period.

8. The computer-implemented method of claim 1, wherein the physiological prediction for the user comprises a plurality of predicted average sensor values for the user during a future time period subsequent to an initial time period corresponding to the plurality of recorded sensor values.

9. The computer-implemented method of claim 8, wherein the future time period comprises a seventy five day time period and the plurality of predicted average sensor values comprises a predicted daily average sensor value for one or more days of the seventy five day time period.

10. The computer-implemented method of claim 1, wherein the machine learning model is previously trained on a labeled training dataset comprising a plurality of historical combined input feature vectors and a plurality of corresponding historical recorded sensor values.

11. The computer-implemented method of claim 10, wherein the labeled training dataset is updated with the combined input feature vector and a plurality of corresponding future recorded sensor values for the user.

12. A computing system comprising memory and one or more processors communicatively coupled to the memory, the one or more processors configured to:
receive one or more initial physiological features for a user that are based on a plurality of recorded sensor values for the user;
generate one or more activity encodings for the user based on a plurality of interaction data objects for the user;
generate a combined input feature vector for the user by aggregating the one or more initial physiological features and the one or more activity encodings;
generate, using a machine learning model, a physiological prediction for the user based on the combined input feature vector; initiate the performance of a prediction-based action based on the physiological prediction; and
initiate the performance of a prediction-based action based on the physiological prediction.

13. The computing system of claim 12, wherein the one or more initial physiological features comprise an aggregated glucose value and an aggregated glucose variability value.

14. The computing system of claim 13, wherein the aggregated glucose value comprises an arithmetic average of a plurality of daily median recorded sensor measurements for the user over an initial time period.

15. The computing system of claim 13, wherein the aggregated glucose variability value comprises an arithmetic average of a plurality of daily median glycemic variability measurements for the user over an initial time period.

16. The computing system of claim 12, wherein the one or more activity encodings comprise:
(i) an event encoding comprising a first one-hot encoding indicative of a presence or an absence of one or more events for the user during a historical time period preceding an initial time period corresponding to the plurality of recorded sensor values, and
(ii) a medication usage encoding comprising a second one-hot encoding indicative of an insulin usage pattern for the user during the historical time period.

17. The computing system of claim 16, wherein each of the plurality of interaction data objects comprises one or more activity codes and the presence or the absence of the one or more events is based on the one or more activity codes.

18. One or more non-transitory computer-readable storage media including instructions that, when executed by one or more processors, cause the one or more processors to:
receive one or more initial physiological features for a user that are based on a plurality of recorded sensor values for the user;
generate one or more activity encodings for the user based on a plurality of interaction data objects for the user;
generate a combined input feature vector for the user by aggregating the one or more initial physiological features and the one or more activity encodings;
generate, using a machine learning model, a physiological prediction for the user based on the combined input feature vector; and
initiate the performance of a prediction-based action based on the physiological prediction.

19. The one or more non-transitory computer-readable storage media of claim 18, wherein the physiological prediction for the user comprises a plurality of predicted average sensor values for the user during a future time period subsequent to an initial time period corresponding to the plurality of recorded sensor values.

20. The one or more non-transitory computer-readable storage media of claim 19, wherein the future time period comprises a seventy five day time period and the plurality of predicted average sensor values comprises a predicted daily average sensor value for one or more days of the seventy five day time period.
